(19) United States
(12) Patent Application Publication
(10) Pub. No.: US 2024/0000538 A1
(43) Pub. Date: Jan. 4, 2024

(54) 3D OUTPUT DEVICE FOR STEREOSCOPIC IMAGE REPRODUCTION

(71) Applicant: Blazejewski MEDI-TECH GmbH, Sexau (DE)

(72) Inventor: Reinhold BLAZEJEWSKI, Gutach i. Br. (DE)

(73) Assignee: Blazejewski MEDI-TECH GmbH, Sexau (DE)

(21) Appl. No.: 18/038,765

(22) PCT Filed: Nov. 29, 2021
(86) PCT No.: PCT/DE2021/200222

§ 371 Date: May 25, 2023

(30) Foreign Application Priority Data
Nov. 30, 2020 (DE) 10 2020 131 595.3

(51) Int. Cl. A61B 90/50; H04N 13/344

(52) U.S. Cl. CPC A61B 90/50; H04N 13/344; A61B 2090/502; A61B 2090/508

(57) ABSTRACT

A 3D output device for stereoscopic image reproduction has an articulated arm with a base joint, via which the articulated arm is movably received on an articulated arm base, and a head joint. A visualization device is arranged movably on the articulated arm by the head joint. A left monitor and a right monitor are arranged in a monitor housing of the visualization device having a left viewing window, which is assigned to the left monitor and through which the left monitor can be viewed from the outside, and a right viewing window, assigned to the right monitor, through which the right monitor can be viewed from the outside, wherein the distance between the left viewing window and the right viewing window corresponds to a typical interocular distance of a person.

The invention relates to a 3D output device for stereoscopic image reproduction. 3D imaging systems can be found, for example, in 3D endoscopes or 3D microscopes. They are used to examine and process objects and structures in medicine, biology, engineering and materials science. The camera or image sensor is often an image converter chip, such as CMOS or CCD. The image sensor, also called an imager, converts the optical signals into electrical signals, which are then made optically visible on a screen or monitor for a person examining the object. To create a three-dimensional image, the 3D imaging system separately records image data for the person's left and right eyes. Typically, a left image channel and a right image channel are used.

A 3D output device is used to visualize the image data acquired by the 3D imaging system in such a way that the person receives a three-dimensional impression of the object under examination. For this purpose, the image data for the left eye and the image data for the right eye must be displayed. The image data intended for the left eye shall be perceived by the subject's left eye only. The image data intended for the right eye shall be perceived only by the right eye of the person. Various devices are known for this purpose, such as 3D monitors that display both the image data for the left eye and the image data for the right eye. The person examining the object usually needs special glasses so that the image data intended for the left eye is perceived only by the left eye and the image data intended for the right eye is perceived only by the right eye of the person. Such glasses include polarizing filter glasses, color filter glasses, interference filter glasses, and LCD shutter glasses. There are also special viewing devices that are placed on a person's head in close proximity to the eyes. Such viewing devices are integrated into a 3D headset, for example. They are also known as 3D video glasses and are equipped with two displays.

A disadvantage of the 3D monitors known in the prior art is that, due to their size, they must be physically separated from the location of the imaging system. Therefore, it is impossible for a person to view both the location of the imaging system and the 3D monitor without turning their head and changing their viewing direction. In addition, the person is limited by the need to wear additional glasses, such as polarizing filter glasses or the like, because the light intensity is reduced. This is particularly distracting when viewing the scene, manually operating the imaging system, and controlling any additional instruments.

A disadvantage of 3D headsets or 3D video glasses is that they are attached to the person's head and cover the eyes. As long as the viewing device is on the person's head, the person cannot see the imaging system's operating area, the imaging system itself, or any ancillary instruments. If the person wishes to remove the viewing device, the person must interrupt the examination of the object or ask a third person to remove the viewing device from the person's head.

Based on the prior art, the object of the invention is to provide a 3D output device for stereoscopic image reproduction that allows a person to see both the location of the imaging system and the 3D image of the examined object captured by the imaging system without significantly changing the viewing direction.

This object is solved by a 3D output device for stereoscopic image reproduction having the features of claim 1. The output device is characterized by an articulated arm and a visualization device. The articulated arm is provided with at least one articulated arm section, a base joint and a head joint. The base joint can have one or more degrees of freedom. The same applies to the head joint. Through the base joint, the articulated arm is movably received on an articulated arm base. The articulated arm base allows the output device to be positioned in a room, for example, by means of a mobile or otherwise movable base, or by means of an attachment to a wall, ceiling, or floor of a room, or to an object in the room. For example, the articulated arm base may be attached to a structure used for examination with a 3D endoscope or with a 3D microscope. Using the head joint, the visualization device is movably arranged on the articulated arm. The visualization device comprises a monitor housing in which a left monitor and a right monitor are arranged. The monitor housing is provided with a left viewing window associated with the left monitor, through which the left monitor can be viewed from the outside. The monitor housing also has a right viewing window associated with the right monitor through which the right monitor can be viewed from the outside. The distance between the left viewing window and the right viewing window corresponds to a typical interocular distance of a person. When a person places his head against the monitor housing, his left eye can view the left monitor through the left viewing window and his right eye can view the right monitor through the right viewing window.

Optical components such as lenses or prisms can be placed between the left viewing window and the left monitor. The same applies to the space between the right viewing window and the right monitor.

The left and right monitors may consist of two spatially separate and distinct monitors. Alternatively, the left and right monitors may be portions of a single monitor.

The visualization device may be coupled to any imaging system that generates three-dimensional image data of an object. In particular, the visualization device may be coupled to a 3D video endoscope or to a 3D microscope. The respective imaging system generates image data for the left eye and for the right eye. This image data is output to the visualization device via an interface such that the image data for the left eye is displayed on the left monitor and the image data for the right eye is displayed on the right monitor. Thus, the person examining the object receives a three-dimensional image of the object from the visualization device. Since the image data for the left eye is displayed on the left monitor and the image data for the right eye is displayed on the right monitor, and since the image information for the left and right eyes is displayed separately by the visualization device provided with viewing windows, there is no need for the user to wear additional glasses such as polarizing filter glasses, color filter glasses, interference filter glasses, or LCD shutter glasses. Compared to known output devices in which all image data is displayed on a single monitor, the output device according to the invention thus has the advantage that the person does not have to wear additional glasses that filter the image data for the right and left eyes accordingly. The person's view of all other areas outside the monitor, such as the site of use of a 3D video endoscope and any instruments used in conjunction therewith, is thus not impaired in terms of light intensity. Furthermore, the absence of additional special glasses means increased comfort for the person and also allows spectacle wearers to use the visualization device.

The articulated arm, which is provided with a base joint and a head joint, allows the visualization device to be adjusted and aligned at the place of use. A person can use the articulated arm and the two joints to adjust the visualization device to their body size and posture so that they can comfortably look through the left viewing window with their left eye and through the right viewing window with their right eye. In addition, the output device can automatically adjust to the person's body size and posture. Once the position of the visualization device is adjusted by the articulated arm, the articulated arm can be fixed. This ensures that the position of the visualization device is maintained even if the user or a third person inadvertently touches the visualization device or the articulated arm.

The person receives the image information displayed on the left monitor directly to the left eye and the image information displayed on the right monitor directly to the right eye. If the imaging system is a 3D video endoscope that is manually operated by the person together with an instrument, the person can look both at the point of use of the endoscope and instrument and through the two viewing windows of the visualization device on the first and second monitor. In order to see both, the person only needs to turn the head slightly. To do this, the visualization device can be adjusted so that the posture and viewing direction of the person has to be changed only slightly to switch between the location of the endoscope and instrument on one side and the left and right monitors on the other side. In this case, the visualization device is held by the articulated arm so that once the position and orientation of the visualization device has been adjusted, the person's hands are free to operate at the point of use. Thus, compared to a single monitor mounted on a wall or other fixture on which all image data for the right and left eyes is displayed together, the output device according to the invention has the advantage that the person does not have to change his or her posture and viewing direction, or at most only slightly, in order to switch between the view of an operation location of an imaging system and the view of the visualization device.

Because the visualization device is attached to the articulated arm and not to the person's body, the person can move away from the visualization device at any time and/or the visualization device can be removed from the person's field of view. It is also possible for the person to look past the visualization device for a moment, for example, to visually check that an instrument is correctly positioned. Once the check is complete, the person can immediately look back at the visualization device. Thus, compared to a visualization device attached to the person's head, the output device according to the invention has the advantage that the person can take the eyes off the left and right monitors without having to remove the output device from the head with the hands.

The articulated arm and/or visualization device can be adjusted either manually or with the aid of an electric drive. For purely manual adjustment, the person adjusts the articulated arm and/or visualization device with the hands to optimize the position for the particular application. If the 3D output device is provided with an electric drive, the person can move the articulated arm and/or visualization device to the desired position by selectively turning the drive on and off. If the electric drive is provided with a controller and sensors or encoders that detect the person or certain parts of the person's body, the 3D output device can also be adjusted automatically. In this case, the person does not need to touch the articulated arm or the visualization device or, if applicable, an input device to adjust the output device. In this case, the adjustment can be done contactlessly.

Advantageously, the 3D output device is combined with a manually operated imaging device. However, it can also be a component of a surgical assistant robot.

According to an advantageous embodiment of the invention, the monitor housing has a viewing window housing wall in which the left and right viewing windows are arranged. The viewing window housing wall has a curvature adapted to the shape of a person's head. This shape of the viewing window housing wall reduces stray light when a person looks through the two viewing windows while bringing his head close to the viewing window housing wall. When the person rests his head against the viewing window housing wall while looking at the monitors through the two viewing windows, the viewing window housing wall assists the person in keeping his head steady.

According to a further advantageous embodiment of the invention, the viewing window housing wall has a nose trough between the left and right viewing windows. Thus, the person can bring his eyes directly to the two viewing windows without interference from his nose.

According to a further advantageous embodiment of the invention, the monitor housing is additionally provided with at least one stray light protection shield which prevents or at least reduces the penetration of stray light into the left and right viewing windows. The stray light protection shield may, for example, be arranged on the viewing window housing wall and protrude laterally beyond the monitor housing. When a person places his or her head against the viewing window housing wall and looks through the two viewing windows, the at least one stray light protection shield will rest against the person's head in the area of the forehead and temples. This may be a continuous stray light protection shield that provides stray light shielding on three sides of the person's head. Alternatively, three separate stray light protection shields may be provided: one for the forehead area and two for the sides of the head. The stray light protection shield may be made of a soft, rubber-like material so that it deforms when the head is placed against it. Injuries to the person are thus avoided.

According to a further advantageous embodiment of the invention, the two viewing windows are provided with eye cups which additionally prevent stray light from entering the monitor housing when a person looks through the left and right viewing windows.

According to a further advantageous embodiment of the invention, the monitor housing has a left housing section provided with the left monitor and with the left viewing window. Further, the monitor housing has a right housing section provided with the right monitor and with the right viewing window. The left housing section is separated from the right housing section in such a way that only the left monitor can be viewed through the left viewing window and only the right monitor can be viewed through the right viewing window. This ensures that a person looking through the two viewing windows sees with the left eye only the information intended for the left eye and with the right eye only the information intended for the right eye. The left and right housing sections can be separated by a divider bar in the monitor housing, for example. It is also possible that the visualization device is binocular and the left housing section is physically separated from the right housing section. In this case, the two housing sections are designed as two separate housing parts.

According to a further advantageous embodiment of the invention, the articulated arm base is height adjustable so that the 3D output device can be adapted to a person's height.

According to a further advantageous embodiment of the invention, the 3D output device has at least three rotational degrees of freedom and one translational degree of freedom for aligning the visualization device in space.

According to a further advantageous embodiment of the invention, the head joint comprises a head pivot joint by means of which the visualization device is mounted on the articulated arm so as to be movable about an axis of rotation. In this case, the visualization device always aligns itself horizontally according to its weight. When the visualization device is aligned horizontally, a straight line extends horizontally through the center of the left viewing window and through the center of the right viewing window. If the visualization device is deflected from the horizontal orientation by an external force, the visualization device will automatically return to the horizontal orientation after the external force is removed. The horizontal orientation is a stable equilibrium position of the visualization device. Advantageously, the visualization device is connected to the head joint such that its center of gravity is below an axis of rotation of the head pivot joint.

According to a further advantageous embodiment of the invention, the head pivot joint comprises a pin arranged on the articulated arm and a bearing shell arranged on the visualization device, surrounding the pin. Alternatively, the pin may be arranged on the visualization device and the bearing shell surrounding the pin may be arranged on the articulated arm.

According to a further advantageous embodiment of the invention, the head joint has, in addition to the pivot joint, at least one further joint with which the inclination of the visualization device can be adjusted when the two viewing windows are aligned horizontally. By adjusting the inclination, the orientation of the visualization device can be adapted to the viewing direction of the person. Advantageously, this adjustment can be locked so that the adjustment does not change unintentionally.

According to a further advantageous embodiment of the invention, the articulated arm comprises at least two articulated arm sections which are movably connected to each other by an intermediate pivot joint. The intermediate pivot joint may have one or more degrees of freedom so that the articulated arm sections can be rotated and tilted in space. The movement of the articulated arm sections adjusts the height and orientation of the visualization device to the position of the person. Compared to an articulated arm with only one articulated arm section, an articulated arm with two articulated arm sections has the advantage of providing more adjustment options. The two sections can have different shapes and/or be made of different materials. For storage or transportation of the 3D output device, the articulated arm can be folded so that the two sections of the articulated arm lie against each other. In this setting, the articulated arm takes up very little space. In use, for example, a first articulated arm section may be oriented vertically upward and a second articulated arm section may be oriented at right angles to the first articulated arm section.

According to another advantageous embodiment of the invention, the articulated arm base is movable. The mobile articulated arm base may be a mobile base such as a wheeled frame or trolley. This allows the 3D output device to be quickly and easily moved from one location of use to another location of use.

According to a further advantageous embodiment of the invention, the articulated arm base is provided with a fastening device with which the articulated arm can be fastened to a wall or a ceiling or a floor of a room. Furthermore, the articulated arm base can also be attached directly to a table, in particular an operating table or a surgical robot, by means of the attachment device. The articulated arm may be detachable from the fastening device.

According to a further advantageous embodiment of the invention, it is provided with at least one drive which adjusts the orientation of the articulated arm and/or the visualization device. The drive comprises, for example, one or more electric motors.

According to a further advantageous embodiment, the drive is provided with a control device. The control device adjusts the articulated arm and the visualization device as a function of predetermined variables or variables detected by sensors or transducers. For example, the natural posture and the viewing direction of a person can be detected, and the 3D output device can be automatically adjusted accordingly. For example, if the person is looking at an endoscope that is to be guided by the person, that viewing direction will be maintained when viewing the 3D image. The drive can also be programmed to follow the control device so that the articulated arm and/or visualization device positions and aligns according to certain specifications. The articulated arm and/or the visualization device are moved by the drive such that a person does not need to touch them during alignment.

According to a further advantageous embodiment of the invention, the control device comprises at least one sensor which collects measurement data and transmits them to the control device. For example, optical sensors can be used. For example, optical sensors can be used to detect markings on the person or in the room. In this case, the articulated arm and/or the visualization device can also be moved without a person having to touch it.

According to a further advantageous embodiment, the control device comprises a voice control. A person's voice commands are picked up by a microphone and converted into corresponding control signals of the drive.

According to a further advantageous embodiment, the control device comprises a motion control. The movements of a person are detected and evaluated in order to control the drive. Preferably, sensors are used to detect the movements.

According to a further advantageous embodiment of the invention, the control device comprises an input device which can be operated manually by a person. Among other things, the input device can be provided with control buttons and a control lever. In this case, the adjustment of the articulated arm and the visualization device is performed by the input of corresponding commands by a person.

The adjustment of the articulated arm and the visualization device may also be performed purely manually by a person applying manual force. Manual control can also be performed by the person directly touching and guiding the articulated arm and/or the visualization device. For example, by placing his head against the monitor housing and pushing it forward.

According to a further advantageous embodiment of the invention, the 3D output device comprises a locking device with which a set alignment of the articulated arm and/or the visualization device can be locked. The locking device can be used to lock the base joint, the head joint and, if necessary, an intermediate pivot joint. The joints can be locked individually or together. The same applies to the reverse case, for example the movable state of the joints. The joints can be locked for example by compressed air. Locking and unlocking can be triggered by a switch or push button located on the monitor housing of the visualization device and can be operated manually. Alternatively, a switch or button may be provided on the floor and operated by foot.

According to a further advantageous embodiment of the invention, the articulated arm is provided with a handle for manually adjusting the orientation of the articulated arm. Handles may be provided on each section of the articulated arm. The visualization device may also be provided with a handle. In sterile environments, it is critical that only certain surfaces are touched. A handle ensures this. The rest of the articulated arm and visualization device remain sterile.

According to a further advantageous embodiment of the invention, the articulated arm and/or the visualization device is provided with at least one replaceable hygiene attachment. The hygiene attachment is made, for example, of a silicone material. The hygiene attachments are provided in particular at the touch points, such as handles, control levers and support surfaces. To ensure sterility, the hygiene attachments are cleaned, disinfected, sterilized or replaced after each use. For example, easily adhered or attached rigid or flexible silicone components are provided. These can also be screwed to the contact surfaces. In addition, silicone components yield to pressure and conform to the contours of the person, providing additional comfort. The other parts of the output device are usually enclosed in a sterile sleeve or membrane and are not touched.

According to a further advantageous embodiment of the invention, the 3D output device is provided with an imaging system. In the medical field, imaging systems are in particular endoscopes, microscopes, laparoscopes, ultrasound devices, X-ray devices or other medical imaging systems. The imaging system captures images for a person's left eye using a left optical channel and images for a person's right eye using a right optical channel. The images are transmitted to the visualization device. For this purpose, supply lines can be provided inside the articulated arm in the direction of the visualization device. Alternatively, the captured images can be transmitted to the visualization device via interfaces, such as wireless interfaces. The latter is particularly useful when the captured images are to be transmitted to a visualization device that is not located in the same room as the object being examined, or when they are to be transmitted to multiple visualization devices.

Further advantages and advantageous embodiments of the invention can be obtained from the following description, the drawing and the claims.

### DRAWING

The drawing shows an embodiment of the subject matter of the invention. Illustrations:

FIG. 1 3D output device in a perspective view,

FIG. 2 Visualization device and a section of the articulated arm of the 3D output device according to FIG. 1,

FIG. 3 3D output device according to FIG. 1 positioned on a table in a perspective view,

FIG. 4 3D output device according to FIG. 1 with a visualization device pivoted to the side in a side view,

FIG. 5 3D output device according to FIG. 4 in a top view,

FIG. 6 3D output device according to FIG. 1 with a rotated visualization device in a view from the side,

FIG. 7 3D output device according to FIG. 6 in a top view,

FIG. 8 3D output device according to FIG. 1 with an articulated arm section of the articulated arm pivoted, in a side view,

FIG. 9 3D output device according to FIG. 8 in a top view,

FIG. 10 3D output device according to FIG. 1 with the visualization device tilted upwards in a side view,

FIG. 11 3D output device according to FIG. 10 in a top view,

FIG. 12 Visualization device of the 3D output device according to FIG. 1 in a perspective view,

FIG. 13 Visualization device of the 3D output device according to FIG. 1 in a view from the front,

FIG. 14 Visualization device of the 3D output device according to FIG. 1 in a side view,

FIG. 15 Visualization device of the 3D output device according to FIG. 1 in a top view,

FIG. 16 3D output device according to FIG. 1 with a person sitting at a table in a view from the side;

FIG. 17 3D output device according to FIG. 1 with a person standing at a table in a view from the side.

### DESCRIPTION OF THE EMBODIMENT

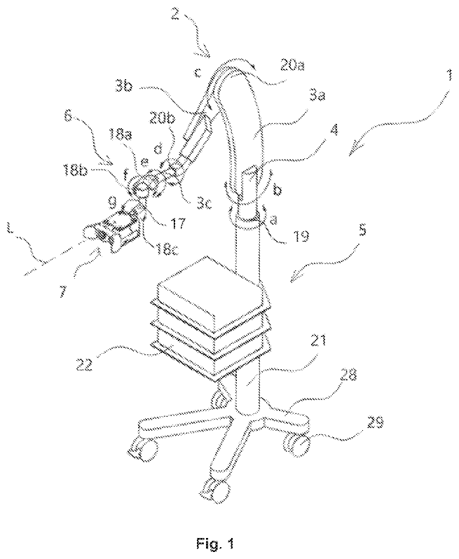

FIG. 1 shows a perspective view of a 3D output device 1 having an articulated arm 2 and a visualization device 7 movably mounted on the articulated arm 2. The articulated arm 2 is movably mounted on an articulated arm base 5. The articulated arm base 5 is a mobile equipment cart with lockable double pivot castors 29. The articulated arm base 5 has a column 21 with a base 28 and a plurality of double pivot castors 29 arranged on the base 28, at least one of which is lockable. A shaft 19 is received on the column 21 of the articulated arm base 5 so as to be adjustable in height and rotatable about its longitudinal axis. This shaft is a component of the articulated arm base 5. The rotational movement of the shaft 19 relative to the column 21 is indicated by the letter a. When the shaft 19 is moved up or down, the articulated arm 2 moves up or down accordingly. In this way, the articulated arm 2 can be adjusted to a user's height. A plurality of equipment plates 22 are arranged on the column 21 for storing equipment and instruments, in particular imaging instruments. The articulated arm 2 comprises three articulated arm sections, namely the first articulated arm section 3a, the second articulated arm section 3b and the third articulated arm section 3c.

One end of the first articulated arm section 3a is movably connected to the articulated arm base 5 via a base joint located at the position marked with reference number 4 in FIG. 1. The base joint is a pivot joint with one degree of freedom. For this purpose, the shaft 19 has, at its end facing the first articulated arm portion 3a, a fork-shaped receptacle in which the first articulated arm section 3a is received so as to be rotatable about an axis not visible in the drawing. This axis constitutes a second axis of rotation of the articulated arm 2. The rotational movement of the base joint is indicated by the letter b.

The end of the first articulated arm section 3a facing away from the base of the articulated arm is movably connected to an end of a second articulated arm section 3b via a first intermediate pivot joint. The position of the first intermediate pivot joint is indicated by reference number 20a. The first intermediate pivot joint forms a third pivot axis of the articulated arm 2. The rotation of the first intermediate pivot joint is indicated by the letter c. The geometric axes of rotation of the base joint and the first intermediate pivot joint are parallel. It follows that the first articulated arm section 3a and the second articulated arm section 3b move in a common plane.

The end of the second articulated arm section 3b facing away from the first articulated arm section 3a is movably connected to one end of a third articulated arm section 3c via a second intermediate pivot joint. The position of the second intermediate pivot joint is indicated by reference number 20b. The second intermediate pivot joint forms a fourth pivot axis of the articulated arm 2, and the rotational movement of the second intermediate pivot joint is indicated by the letter d. The geometric axis of rotation of the second intermediate pivot joint is perpendicular to the geometric axes of rotation of the base pivot joint and the first intermediate pivot joint. A movement of the third articulated arm section 3c relative to the second articulated arm section 3b about the second intermediate pivot joint does not result in a change of the angle between these two articulated arm portions, but in a rotation of the third articulated arm portion 3c about its longitudinal axis.

In accordance with the base joint, the first intermediate pivot joint and the second intermediate pivot joint are pivot joints with one degree of freedom.

The end of the third articulated arm section 3c facing away from the second articulated arm section 3b is connected to the visualization device 7 by a head joint. The position of the head joint is indicated by reference number 6. The head joint comprises a first head pivot joint 18a, a second head pivot joint 18b, a third head pivot joint 18c, and a coupling member 17 connecting the second head pivot joint 18b to the third head pivot joint 18c. The first, second and third head pivot joints 18a, 18b, 18c form fifth, sixth and seventh pivot axes of the articulated arm 2. A rotational movement enabled by the first head pivot joint 18a is indicated by the letter e. A rotational movement enabled by the second head pivot joint 18b is indicated by the letter f. A rotational movement enabled by the third head pivot joint 18c is indicated by the letter g. The geometric axes of rotation of the first head pivot joint 18a and the second head pivot joint 18b are perpendicular to each other. Here, the geometric rotational axis of the first head pivot joint 18a is horizontal and the geometric rotational axis of the second head pivot joint 18b is vertical. The first head pivot joint 18a and the second head pivot joint 18b each have one degree of freedom.

The coupling member 17 has the second head pivot joint 18b at its first end and the third head pivot joint 18c at its second end opposite to the first end. The third head pivot joint 18c allows the visualization device 7 to be rotated about the longitudinal axis L of the present elongated visualization device 7. The longitudinal axis L of the visualization device 7 is represented by a dotted line.

In total, the articulated arm 2, with the pivot joint of the articulated arm base 5, the base joint, the first head pivot joint 18a, the second head pivot joint 18b, the third head pivot joint 18c, the first intermediate pivot joint and the second intermediate pivot joint, has seven axes of rotation which allow the adjustment of the visualization device 7. In addition, a linear degree of freedom is provided by the shaft 19 which is received in the column 21 in a height-adjustable manner.

The articulated arm sections 3a, 3b, 3c of the articulated arm 2 and the visualization device 7 are moved and aligned by manual force transmission by a person or supported by a drive. Such a drive is not shown in the drawing. In this case, all or only some of the joints of the articulated arm 2 are moved in order to move the articulated arm 2 and the visualization device 7 from a first position to a second position. In a set position, the joints of the articulated arm 2 can be locked by means of a locking device not shown in FIG. 1.

When the first articulated arm section 3a is moved by the base joint, an acute or obtuse angle is formed between the column 21 and the first articulated arm section 3a. When the base joint of the articulated arm 2 is locked by compressed air, the first articulated arm section 3a remains in a fixed position until the locking of the base joint is released and the first articulated arm section 3a is moved. The same applies to the other joints and articulated arm sections 3b, 3c of the articulated arm 2.

If the first intermediate pivot joint is locked by compressed air but the base joint is not, the angle that the first articulated arm section 3a and the second articulated arm section 3b enclose with each other remains the same even if the first articulated arm section 3a is moved by the base joint. In this case, the second articulated arm section 3b is moved by the base joint. When all joints of the articulated arm 2 are locked, the articulated arm 2 and the visualization device 7 will not move even if the articulated arm 2 or the visualization device 7 is accidentally touched by a user.

The seven rotation axes of the articulated arm 2 make it possible to align the visualization device 7 quickly and easily, even if complex movements of the articulated arm 2 are to be performed.

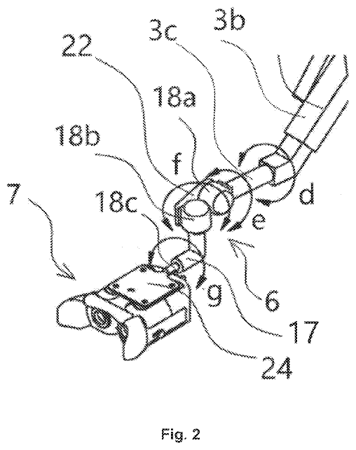

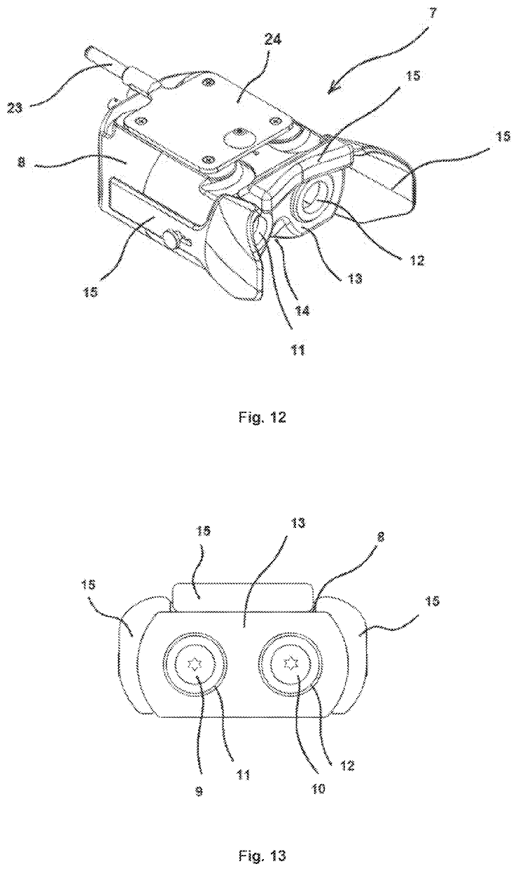

FIG. 2 shows a detailed view of the head joint 6. The head joint 6 comprises the first head pivot joint 18a, the second head pivot joint 18b and the third head pivot joint 18c. The first head pivot joint 18a is fixedly connected to the second head pivot joint 18b by the connector 22. Rotation about the axis of rotation of the first head pivot joint 18a causes the connector 22 and the second head pivot joint 18b to move up or down. The second head pivot joint 18b receives one end of the coupling member 17 so that it can be rotated by the second head pivot joint 18b. The end of the coupling member 17 facing away from the second head pivot joint 18b receives the third head pivot joint 18c, through which it is movably connected to the visualization device 7. For this purpose, a plate 24 is provided on the visualization device 7 wherein the plate is firmly screwed to the visualization device. The details are shown in FIG. 12. In order to align the visualization device 7 three-dimensionally in space, the articulated arm sections 3a, 3b, 3c as well as the visualization device can be moved around the various pivot joints according to the arrows b, c, d, e, f and g.

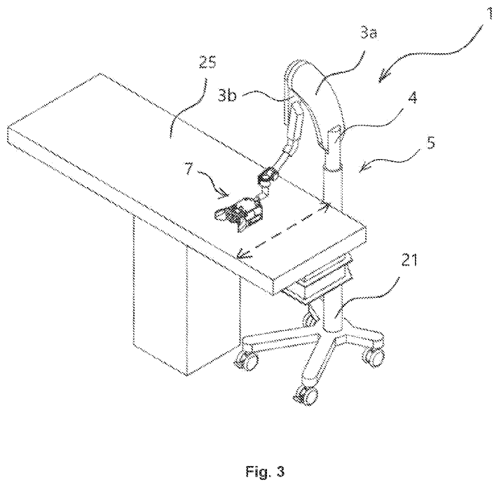

FIG. 3 shows the 3D output device 1 of FIG. 1 arranged on a table 25. The articulated arm base 5 is arranged on the floor so that the column 21 of the articulated arm base is arranged partly below and partly above the table 25. The visualization device 7 is arranged above the table 25. When the first articulated arm section 3a is moved, the base joint disposed at the position indicated by reference number 4 causes the first articulated arm section 3a to approach or move away from the surface of the table 25. When the second articulated arm section 3b is moved relative to the first articulated arm section 3a, the visualization device 7 moves in the direction of the dotted double arrow. At present, the upper surface of the visualization device 7 is arranged horizontally with respect to the surface of the table 25 at a distance therefrom.

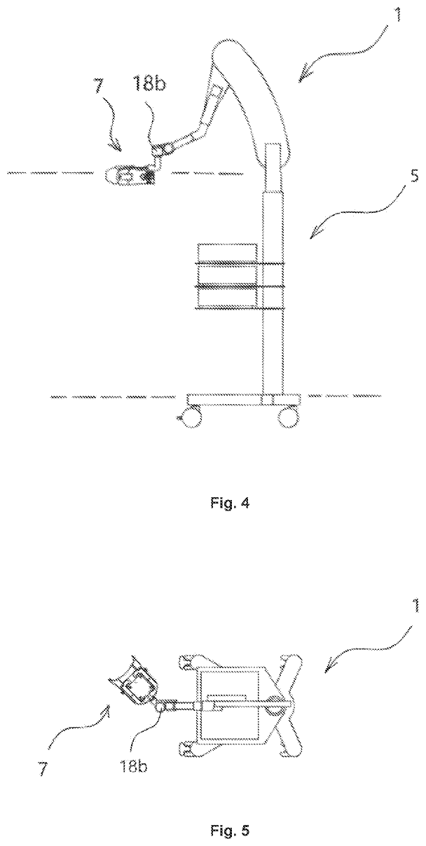

FIGS. 4 and 5 show the 3D output device 1 of FIG. 1 with a horizontally oriented visualization device 7 pivoted to one side in a side view and in a top view. The horizontal orientation of the visualization device 7 is indicated by two dashed lines, one representing the orientation of the floor on which the articulated arm base 5 is arranged and the other representing the orientation of the visualization device 7. The lateral pivoting movement of the visualization device 7 is caused by a movement of the coupling member 17 about the first head pivot joint 18b. In particular, the visualization device 7 is pivoted to the side when it is to be temporarily removed from a person's field of view.

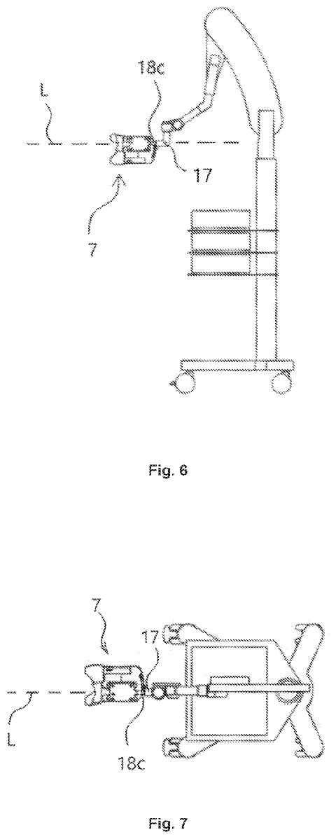

FIGS. 6 and 7 show the 3D output device of FIG. 1 with a visualization device 7 rotated about the axis of rotation of the third head pivot joint 18c in a plan view from the side. The axis of rotation of the third head pivot joint 18c is arranged coaxially with the longitudinal axis L of the visualization device 7. The third head pivot joint 18c is arranged at the end of the coupling member 17 facing the visualization device 7. In contrast to the previous figures, the upper side of the visualization device 7 is no longer aligned horizontally with respect to the ground. When the third head pivot joint 18c is locked, the visualization device 7 remains in this position, rotated about the axis of rotation of the third head pivot joint 18c. When the locking of the third head pivot joint 18c is released, the upper side of the visualization device 7 rotates independently by following the gravitational force back to its equilibrium position horizontally aligned with the ground. An orientation of the visualization device 7 rotated about the axis of rotation of the third head pivot joint 18c is particularly advantageous when an imaging system not shown in FIG. 6 is rotated in an object to be examined and the user wishes to follow the rotation of the imaging system with his gaze.

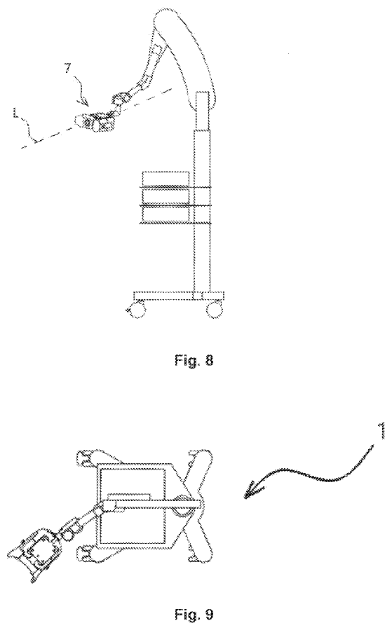

FIGS. 8 and 9 show an embodiment in which the second articulated arm section 3b is additionally received on the first articulated arm section 3a so as to be rotatable about a vertical axis of rotation, or the second articulated arm section 3b itself has an additional pivot joint. FIGS. 8 and 9 show a rotation of the second articulated arm section 3b relative to the first articulated arm section 3a about such a pivot joint.

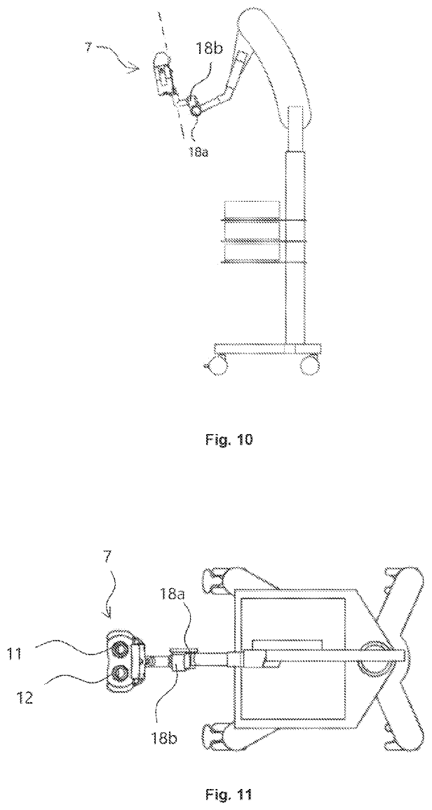

FIGS. 10 and 11 show the 3D output device according to FIG. 1 with a visualization device 7 rotated about the axis of rotation of the first head pivot 18a and thus tilted upwardly in a side view and in a top view. The rotation of the visualization device 7 about the axis of rotation of the first head pivot joint 18a causes the second head pivot joint 18b to move upwardly so that the second head pivot joint 18b is disposed above the first head pivot joint 18a. The tilted arrangement of the visualization device 7 is particularly advantageous when a person stands upright and looks from above into the left and right viewing windows 11, 12 of the visualization device 7 at an object to be examined.

FIG. 12 shows a perspective view of the visualization device 7. It can be clearly seen that the plate 24 is attached to the top of the visualization device 7 by four screws. The plate is essentially flush with the side edges of the top of the visualization device 7. At the rear end of the plate there is the pin 23 which is rotatably received by the coupling member 17 not shown in FIG. 12. An adjustable and detachable upper stray light protection shield 15 is arranged on the monitor housing 8, which prevents light from above from entering the area of the viewing window housing wall 13. In addition, two further lateral stray light protection shields 15 are provided on the side surfaces of the monitor housing 8 to prevent light from the side from entering the area of the viewing window housing wall 13. The stray light protection shield 15 are formed of a deformable silicone material. The viewing window housing wall 13 is provided with a nose trough 14 located between the left and right viewing windows 11, 12. A person can place his forehead and nose against the viewing window housing wall 13 and does not need to wear special glasses. If the person wears glasses, he or she does not need to take off the glasses when looking through the left and right viewing windows 11, 12. In this way, a person can simultaneously see the object in front of them and the 3D image of the object in focus.

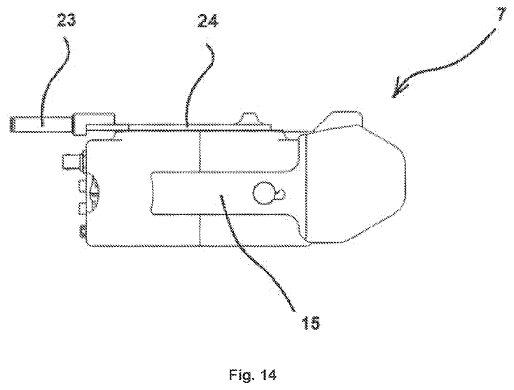

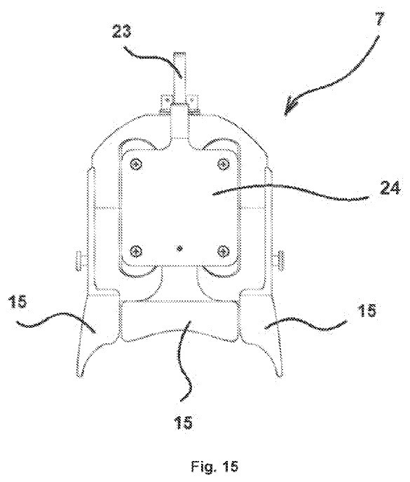

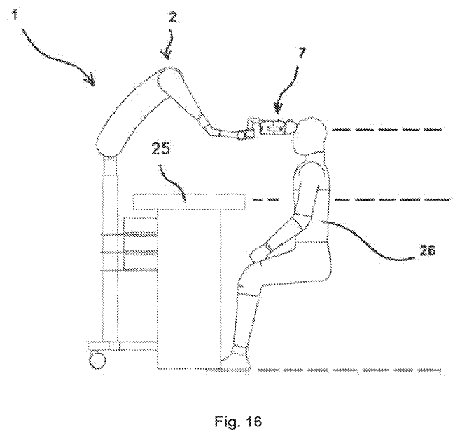

FIGS. 13, 14 and 15 show the visualization device 7 according to FIG. 1 from the front, from the side and from above. In FIG. 13 it can be clearly seen that a structure of the examined object is displayed on the left and right monitors 9, 10. The structure is star-shaped. The left and right monitors 9, 10 are arranged within the monitor housing 8 in extension of the round left viewing window 11 and the likewise round right viewing window 11, 12. The viewing window housing wall 13 is bounded by the stray light protection shield 15. FIG. 13 does not show the nose trough. FIG. 14 shows that the top of the visualization device 7 is attached to the plate 24. This allows the visualization device 7 to be mounted quickly and easily. Via the pin 23 of the plate 24, the visualization device 7 is held by the coupling member 17, which is not shown in FIG. 14. The stray light protection shields 15 are movable in the longitudinal direction of the visualization device 7. FIG. 15 shows that the stray light protection shields 15 are ergonomically adapted to the head shape of a user and project beyond the viewing window housing wall, so that even a user who wears glasses and therefore keeps a certain distance from the left and right viewing windows is not blinded by ambient light.

FIG. 16 shows the 3D output device 1 of FIG. 1 with a person 26 sitting at a table 25 in a side view. The top of the visualization device 7 is oriented horizontally to the surface of the table 25 and to the floor. This is shown by three dotted lines. The person 26 looks straight ahead and sees the outside of an object to be examined and the inside of the object to be examined in the form of a 3D image. The 3D image is captured by an imaging system controlled by the person 26 and transmitted to the visualization device 7. In order to switch between the real working space and the virtual space, the person 26 simply has to look past the visualization device 7 or move it out of his field of vision. The person only has to turn his head slightly. The person 26 moves the visualization device 7, for example, by hand or by means of one or more motors controlled by an input device. It can be clearly seen that the visualization device 7 is not attached to the head of the person 26, but exclusively to the articulated arm 2. The person 26 can thus move away from the visualization device 7 at any time without having to touch it. For example, a movement of the head backwards or sideways is sufficient to disengage from the visualization device 7.

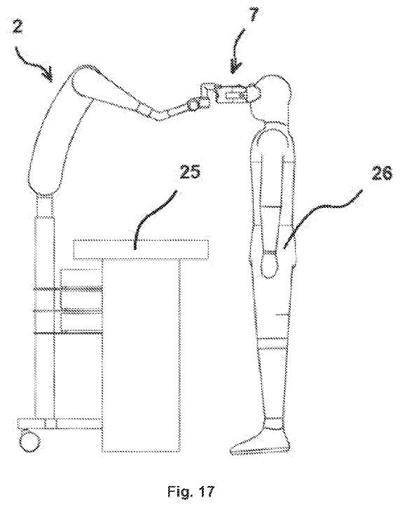

FIG. 17 shows a side view of the 3D output device according to FIG. 1 with a person 26 standing at a table 25. It can be clearly seen that the person 26 can not only move away from the visualization device 7 at any time, but can also adopt a different posture at any time by changing from the sitting position shown in FIG. 9 to the standing position shown in FIG. 17. If the 3D output device 1 is equipped with sensors, the articulated arm 2 and the visualization device 7 can also follow the movements of the person 26. For example, if the person moves his head forward or steps closer to the object, the articulated arm 2 and the visualization device 7 could automatically move backward by the same distance.

All of the features of the invention may be essential to the invention either individually or in any combination.

### REFERENCE NUMBERS

1 3D output device
2 Articulated arm
3
3a First articulated arm section
3b Second articulated arm section
3c Third articulated arm section
4 Position of the base joint
5 Articulated arm base
6 Position of the head joint
7 Vizualization device
8 Monitor housing
9 Left monitor
10 Right monitor
11 Left viewing window
12 Right viewing window
13 Viewing window housing wall
14 Nose trough
15 Stray light protection shield
16
17 Coupling member
18
18a First head pivot joint
18b Second head pivot joint
18c Third head pivot joint
19 Shaft
20a Position of first intermediate pivot joint
20b Position of second intermediate pivot joint
21 column
22 Connector
23 Pin
24 Plate
25 Table
26 Person
27
28 Base
29 Double pivot castors
a Rotation of the joint of the articulated arm base
b Rotation of the head joint
c Rotation of the first intermediate pivot joint
d Rotation of the second intermediate pivot joint
e Rotational movement of the first head pivot joint
f Rotational movement of the second head pivot joint
g Rotational movement of the third head pivot joint

1: A 3D output device (1) for stereoscopic image reproduction,
with an articulated arm (2),
wherein the articulated arm (2) is provided with a base joint, via which the articulated arm (2) is movably received on an articulated arm base (5), and wherein the articulated arm (2) is provided with a head joint,
with a visualization device (7), which is arranged movably on the articulated arm (2) by the head joint,
with a monitor housing (8) of the visualization device (7),
with a left monitor (9) and a right monitor (10) which are arranged in the monitor housing (8),
wherein the monitor housing (8) is provided with a left viewing window (11), which is assigned to the left monitor (9) and through which the left monitor (9) can be viewed from the outside,
wherein the monitor housing (8) is provided with a right viewing window (12), assigned with the right monitor (10), through which the right monitor (10) can be viewed from the outside,
wherein the distance between the left viewing window (11) and the right viewing window (12) corresponds to a typical interocular distance of a person.

2: The 3D output device according to claim 1, wherein the monitor housing (8) has a viewing window housing wall (13) in which the left and right viewing windows (11, 12) are arranged, and that wherein the viewing window housing wall (13) has a curvature adapted to the shape of a person's head.

3: The 3D output device according to claim 2, wherein the viewing window housing wall (13) has a nose trough (14) between the left and right viewing windows (11, 12).

4: The 3D output device according to claim 1, wherein the monitor housing (8) is provided with at least one stray light protection shield (15) which reduces the penetration of stray light into the left and right viewing windows (11, 12).

5: The 3D output device according to claim 1, wherein the monitor housing (8) has a left housing section provided with the left monitor (9) and with the left viewing window (11), and wherein the monitor housing (8) has a right housing section provided with the right monitor (10) and with the right viewing window (12), and wherein the left housing section (16) is separated at least in sections from the right housing section (17) in such a way that only the left monitor (9) can be viewed through the left viewing window (11) and only the right monitor (10) can be viewed through the right viewing window (12).

6: The 3D output device according to claim 1, wherein the head joint (6) comprises a head pivot joint (18a, 18b, 18c) by means of which the visualization device (7) is mounted on the articulated arm (2) so as to be movable about an axis of rotation and aligns itself horizontally according to its weight, wherein a straight line extending through the center of the left viewing window (11) and through the center of the right viewing window (12) is horizontal.

7: The 3D output device according to claim 6, wherein the head pivot joint (18a, 18b, 18c) comprises a pin (23) arranged on the articulated arm (2) and a bearing shell arranged on the visualization device (7) surrounding the pin (23).

8: The 3D output device according to claim 1, wherein the articulated arm (2) comprises at least two articulated arm sections (3a, 3b, 3c, 3d) which are movably connected to each other by an intermediate pivot joint (20a, 20b).

9: The 3D output device according to claim 1, wherein the articulated arm base (5) is movable.

10: The 3D output device according to claim 1, wherein the articulated arm base (5) is provided with a fastening device with which the articulated arm (2) can be fastened to a wall or ceiling of a room or to an object.

11: The 3D output device according to claim 1, wherein the 3D output device is provided with at least one drive which adjusts the orientation of the articulated arm (2) and/or of the visualization device (7).

12: The 3D output device according to claim 11, wherein the drive is provided with a control device.

13: The 3D output device according to claim 12, wherein the control device comprises a voice control.

14: The 3D output device according to claim 12, wherein the control device comprises a motion control which detects movements of a user, evaluates the movements and controls the drive accordingly.

15: The 3D output device according to claim 12, wherein the control device is equipped with a manually operable input device.

16: The 3D output device according to claim 1, wherein the 3D output device comprises a locking device with which a set alignment of the articulated arm (2) and/or of the visualization device (7) can be locked.

17: The 3D output device according to claim 1, wherein the articulated arm (2) is provided with a handle for manually adjusting the orientation of the articulated arm (2).

18: The 3D output device according to claim 1, wherein the articulated arm (2) and/or the visualization device (7) is provided with a replaceable hygiene attachment.

19: The 3D output device according to claim 1, wherein the 3D output device is provided with an imaging system.
